(19) United States
(12) Patent Application Publication
(10) Pub. No.: US 2002/0132259 A1
(43) Pub. Date: Sep. 19, 2002

(54) Mutation detection using MutS and RecA

(76) Inventors: Robert E. Wagner JR., Carr, CO (US); Miroslav Radman, Gentilly (FR)

Correspondence Address: Venable, P.O. Box 34385, Washington, DC, 20043-9998, US

(21) Appl. No.: 10/078,278

(22) Filed: Feb. 20, 2002

Related U.S. Application Data
(60) Provisional application No. 60/270,428, filed on Feb. 21, 2001.

(51) Int. Cl. C12Q 1/68; C07H 21/04

(52) U.S. Cl. 435/6; 536/24.3

(57) ABSTRACT

A method for detecting mutations and polymorphisms, including single nucleotide polymorphisms (SNP's), is based on the use of RecA-like recombinase protein and a MutS-like mismatch binding protein. RecA coated, specific DNA oligonucleotide probes (RecA filaments) are used for homology searching in duplex DNA. Location of homologous sequences results in the formation of D-loop structures containing a duplex region comprised of the oligonucleotide probe and one strand of the test DNA. Mismatches or unpaired bases in the duplex region are substrates for MutS binding. Co-localization of MutS and oligonucleotide or RecA labels is diagnostic of specific sequence differences between the probe and test DNAs. Also provided are compositions and kits useful for practicing the methods of the present invention.

### BACKGROUND OF THE INVENTION

1. Field of the Invention

The present invention in the fields of molecular biology and medicine relates to a method for detecting mutations and polymorphisms involving as little as one base change (Single Nucleotide Polymorphism—SNP) or a single base addition to or deletion from the wild-type DNA sequence.

2. Description of the Background Art

Progress in human molecular and medical genetics depends on the efficient and accurate detection of mutations and sequence polymorphisms, the vast majority of which results from single base substitutions (Single Nucleotide Polymorphisms—SNP) and small additions or deletions. Assays capable of detecting the presence of a particular mutation, SNP or mutant nucleic acid sequence in a sample are therefore of substantial importance in the prediction and diagnosis of disease, forensic medicine, epidemiology and public health. Such assays can be used, for example, to detect the presence of a mutant gene in an individual, allowing determination of the probability that the individual will suffer from a genetic disease. The ability to detect a mutation has taken on increasing importance in early detection of cancer or discovery of susceptibility to cancer with the discovery that discrete mutations in cellular oncogenes can result in activation of that oncogene leading to the transformation of that cell into a cancer cell and that mutations inactivating tumor suppressor genes are required steps in the process of tumorigenesis The detection of SNPs has assumed increased importance in the identification and localization (mapping) of genes, including those associated with human and animal diseases.

The desire to increase the utility and applicability of such assays is often frustrated by assay sensitivity as well as complexity and cost. Therefore, it would be highly desirable to develop more sensitive, simple and relatively inexpensive assays for detection of alterations in DNA.

Nucleic acid detection assays can be based on any of a number of characteristics of a nucleic acid molecule, such as its size, sequence, susceptibility to cleavage by restriction endonucleases, etc. The sensitivity of such assays may be increased by altering the manner in which detection is reported or signaled to the observer. Thus, for example, assay sensitivity can be increased through the use of detectably labeled reagents such as enzymes (Kourilsky et al., U.S. Pat. No. 4,581,333), radioisotopes (Falkow et al., U.S. Pat. No. 4,358,535; Berninger, U.S. Pat. No. 4,446,237), fluorescent labels (Albarella et al., EP 144914), chemical labels (Sheldon III et al., U.S. Pat. No. 4,582,789; Albarella et al., U.S. Pat. No. 4,563,417), modified bases (Miyoshi et al., EP 119448), and the like.

Most methods devised to attempt to detect genetic alterations consisting of one or a few bases involve amplification of specific DNA regions by PCR and many involve hybridization between a standard nucleic acid (DNA or RNA) and a test DNA such that the mutation is revealed as a mispaired or unpaired base in a heteroduplex molecule. Detection of these mispaired or unpaired bases has been accomplished by a variety of methods (Myers, R M, et al., Cold Spring Harbor Symp. Quant. Biol. 51:275-284 (1986); Gibbs, R. et al., Science 236:303-305 (1987); Lu, A S. et al., 1992, Genomics 14:249-255 (1992); Cotton, R G, et al., Proc. Natl. Acad. Sci. USA 85:4397-4401 (1988); Cotton, R G, Nucl. Acids Res 17:4223-4233 (1989); Lishanski, A. et al., Proc. Natl. Acad. Sci. USA 91:2674-2678 (1994); Wagner, R E, et al., Nucl. Acids Res. 23:3944-3948 (1995); Debbie, P. et al., Nucl Acids Res 25:4825-4829 (1997). These methods all suffer from the requirements that: (1) a specific DNA region must be amplified by polymerase chain reaction (PCR) prior to mutation or SNP detection and (2) amplified DNA must be denatured and allowed to anneal with some standard DNA of known genotype in order to allow the formation of mismatches or unpaired bases. PCR amplification introduces errors during amplification (it is a relatively low-fidelity process) and denaturation and annealing, particularly of genomic DNA or of large amplicons, can leave large, un-annealed single stranded fragments which can adopt secondary structures containing regions of double stranded DNA with unpaired or mispaired bases. These mismatched and unpaired bases can interfere with the detection of the target mismatches or unpaired bases. Many of these methods also require that the exact location of the mutation be known and are difficult to interpret when the sample DNA is heterozygous for the mutation in question. Therefore, most are not practical for use in screening for mutations and SNPs.

MutS and RecA are bacterial proteins involved in DNA repair and genetic recombination and have been best characterized in E. coli. MutS is the mismatch recognition and binding protein of the E. coli mismatch repair system, which functions to repair errors made by DNA polymerase during DNA replication. The system also recognizes mismatches in the hybrid overlaps created in the initial steps of genetic recombination and acts on such mismatch containing regions to abort recombination. Thus, the mismatch repair system is an editor both in DNA replication and genetic recombination and assures high fidelity in both processes. (The editing of recombination is essential to avoid chromosomal rearrangements, to allow successful meiosis and to erect a genetic barrier between closely related species.)

MutS has been used in mutation and SNP detection (Lishanski et al., supra; Wagner et al., supra; Debbie et al. supra; patents by Wagner and colleagues—U.S. Pat. Nos. 6,027,877, 6,114,115, 6,120,992, 6,329,147; and Gifford U.S. Pat. No. 5,750,335). When used in solution, as in filter binding or gel shift assays (Jiricny et al., Nucl Acids Res. 16:7843-7853 (1988); Lishanski et al., supra) MutS performs poorly, in that it does not detect most mismatches and exhibits high levels of background binding of non-mismatched DNA. Immobilized MutS has a greatly increased ability to bind mismatches and a greatly diminished ability to bind DNA without mismatches (Wagner et al., supra). However, even immobilized MutS suffers from PCR induced errors in DNA, including both misincorporation and mispriming, and single stranded DNA is a powerful competitor in mutation and SNP detection assays.

RecA, a bacterial recombinase which has been best characterized in E. coli, is the key player in the process of genetic recombination, in particular in the search and recognition of sequence homology, and the initial strand exchange process. RecA can catalyze strand exchange in the test tube. Recombination is initiated when multiple RecA molecules coat a stretch of single stranded DNA (ssDNA) to form what is known as a RecA “filament.” This filament, in the presence of ATP, searches for homologous sequences in double stranded DNA (dsDNA). When homology is located, a three stranded (D-loop) structure is formed wherein the RecA filament DNA is paired with the complementary strand of the duplex. If pairing is not perfect, i.e., if there are mismatches or unpaired bases in the newly created duplex, MutS can bind to these structures and mobilize the other proteins of the mismatch repair system which act to abort the recombination event by removing the filament DNA and restoring the original duplex. Considerable evidence (most of it still unpublished) suggests that RecA and MutS co-localize during recombination and that RecA binding to DNA may facilitate MutS mismatch recognition, perhaps by improving the presentation of mismatches to MutS.

RecA has been used to facilitate screening of plasmid libraries for plasmids containing specific sequences (Rigas et al., Proc Natl Acad Sci USA. 83:9591-9595 (1986)). In this application, biotinylated single stranded DNA probes are reacted with RecA to form RecA filaments. The filaments are used for homology searching in circular plasmid DNA. When the probes are removed by binding to avidin, those plasmids containing sequences homologous to the probes are isolated by virtue of the triple stranded (D-loop) structures formed by the RecA filament and the plasmid duplex. In order for these structures to be stable it is necessary to use adenosine 5′-[γ-thio]triphosphate (ATP[γ-S]) in place of ATP. ATP[γ-S] allows homology searching by RecA, by is non-hydrolyzable and thus does not allow RecA dissociation from the triple stranded structure.

RecA has also been used to facilitate the mapping of specific DNA regions from bacterial and human genomic DNA (Ferrin, L J, et al., Science 254:1494-1497 (1991); Ferrin, L J, et al., Nature Genetics 6:379-383 (1994)). In these applications, RecA is used in conjunction with restriction enzymes (sequence specific double strand DNA endonucleases) to allow isolation or identification of specific DNA fragments. RecA filaments are prepared and reacted with genomic DNA under conditions that allow triple strand (D-loop) structure formation. The DNA is then treated with either a restriction endonuclease or a modification methylase (methylase action transfers a methyl group to the specific recognition sequence of a specific restriction endonuclease, thus protecting the sequence from endonuclease digestion). The presence of the RecA filament in the triple strand structure prevents digestion or methylation.

Specific RecA filaments have also been used to protect restriction endonuclease generated “sticky-ends” from being filled in by DNA polymerase such that, upon removal of the RecA filaments, specific fragments can be cloned into plasmid vectors (Ferrin et al., U.S. Pat. No. 5,707,811).

Formation of RecA catalyzed double D-loops has been used to identify and isolate specific DNA regions from double stranded DNA (Sena et al., U.S. Pat. Nos. 5,273,881 and 5,670,316), This method requires relatively long DNA probes (>78 nucleotides) and complementarity between the probes and double D-loops in order to provide for a stable structure. This contrasts fundamentally from the present invention wherein the probe and test DNA that together form a D loop must have sequence differences that result in formation of mispaired or unpaired bases in a probe/test duplex region (to allow detection of a mutation or SNP). These documents note the possibility of introducing a detectable label into the probe by oligonucleotide extension with DNA polymerase. Importantly, this method is only suited for detection of specific sequences in a test DNA but is of no use in detecting mutations or SNPs—the object of the present invention. Moreover, the teaching of these patents is limited to use of a double D-loop. There is no suggestion of using of single D-loops, even for the limited purpose of sequence detection.

All statements as to the date or representation as to the contents of these documents is based on the information available to the applicant and does not constitute any admission as to the correctness of the dates or contents of these documents.

### SUMMARY OF THE INVENTION

The present invention is directed to a method for detecting of a mutation and/or a SNP in a double-stranded test DNA molecule, comprising:

(a) providing a single stranded DNA probe which is optionally detectably labeled, which probe has (i) a known nucleotide sequence or (ii) a sequence complementary to the sequence of at least a part of the test DNA.

(b) contacting the probe with a RecA protein (or a homologue thereof, defined in more detail below), which is optionally detectably labeled, to form a RecA filament,

(c) contacting the RecA filament with the test DNA, thereby forming a three stranded DNA D-loop structure in the test DNA, which D-loop structure comprises the probe and the two strands of the test DNA;

(d) contacting the DNA D-loop structure with MutS (or a homologue thereof, defined in more detail below), which is optionally detectably labeled, wherein the MutS binds to one or more base pair mismatches or unpaired bases present in the duplex portion of D-loop structure;

(e) detecting the presence of MutS bound to the DNA D-loop structure

wherein the presence of the bound MutS is indicative of the presence of the mutation or the SNP in the test DNA.

Also provided is a method for detecting a mutation and/or a SNP in a double-stranded test DNA molecule, comprising:

(a) providing a probe comprising two complementary single stranded oligonucleotides which are optionally detectably labeled, which probe has (i) a known nucleotide sequence or (ii) a sequence complementary to the sequence of at least a part of the test DNA;

(b) contacting each of the oligonucleotides in single stranded form with a RecA protein, which is optionally detectably labeled, to form RecA filaments,

(c) contacting the RecA filaments with the test DNA, thereby forming a four stranded DNA structure in the test DNA, which structure comprises the probe and the two strands of the test DNA and wherein the probe strands are annealed with test DNA strands;

(d) contacting said DNA structure with a MutS protein which is optionally detectably labeled, wherein the MutS binds to one or more base pair mismatches or unpaired bases present in the four stranded DNA structure;

(e) detecting the presence of the MutS bound to the DNA structure

wherein the presence of the bound MutS is indicative of the presence of the mutation or the SNP in the test DNA.

Another method for detecting a mutation and/or a SNP in a double-stranded test DNA molecule, comprises:

(a) providing a single stranded DNA probe which is optionally detectably labeled, which probe has (i) a known nucleotide sequence or (ii) a sequence complementary to the sequence of at least a part of the test DNA molecule;

(b) contacting the probe with a RecA protein which is optionally detectably labeled, to form a RecA filament,

(c) contacting the RecA filament with the test DNA, thereby forming a three stranded DNA D-loop structure in the test DNA, which structure comprises the probe and two strands of the test DNA;

(d) contacting the DNA D-loop structure with immobilized MutS which binds to one or more base pair mismatches or unpaired bases present in the duplex portion of the D-loop structure;

(e) detecting the presence immobilized probe DNA or RecA bound to the MutS,

wherein the presence of the bound probe DNA or RecA is indicative of the presence of the mutation or the SNP in the test DNA.

The invention includes a method for detecting a mutation and/or a SNP in a double-stranded test DNA molecule, comprising:

(a) providing a probe comprising two complementary single stranded oligonucleotides which are optionally detectably labeled, which probe has (i) a known nucleotide sequence or (ii) a sequence complementary to the sequence of at least a part of the test DNA;

(b) contacting each of the probe oligonucleotides in single stranded form with a RecA protein, which is optionally detectably labeled, to form RecA filaments,

(c) contacting the RecA filaments with the test DNA, thereby forming a four stranded DNA structure in the test DNA, which structure comprises the two strands of the test DNA to each of which is annealed a probe oligonucleotide strand; and

(d) contacting said DNA structure with immobilized MutS which binds to one or more base pair mismatches or unpaired bases present in said four stranded DNA structure,

thereby detecting the mutation and/or SNP.

In the above method, test DNA molecule may be prokaryotic genomic DNA, eukaryotic genomic DNA, cDNA, viral DNA, plasmid DNA, and a DNA fragment amplified by PCR or by another amplification method.

To affirmatively detect a mutation or SNP, the above probe sequence must differ from the sequence of the mutation or SNP by one or more nucleotide substitutions, additions or deletions such that the probe/test heteroduplex contains mismatched or unpaired bases recognizable by MutS, or a MutS homologue or other mismatch-binding protein (“MBP”). If the test DNA sequence is identical to the probe, the test result is “negative.” It should be understood that “a probe” as used above may include “one or more” different probe molecules. The probe is preferably an oligonucleotide of about 20 to about 60 nucleotides, and is preferably selected from the group consisting of: (a) a synthetic oligonucleotide; (b) a recombinant oligonucleotide; and (c) an oligonucleotide obtained by denaturing, and, optionally, cleaving, a double stranded DNA molecule. When the probe comprises two, complementary DNA molecules, they may be separately coated with RecA. The probe may include an adduct, which may an oligonucleotide, biotin or digoxigenin, or the like, to allow immobilization following D-loop formation.

The RecA protein is preferably from E. coli.

In the methods described herein, detection is based on the use any one of the components detectably labeled: the probe DNA, the RecA, the MutS, (or SSB, discussed below). The label may be any suitable detectable label, e.g., a fluorophore, a chromophore, a radionuclide, biotin, digoxigenin, etc. The protein or DNA may be labeled via a bead to which is attached the above fluorophore, chromophore, biotin, etc. The probe may be labeled by DNA polymerase extension using labeled deoxynucleotide triphosphates or nucleotide terminators.

Preferred detection is of bound MutS which may be in solution or immobilized to a solid surface such as nitrocellulose, polystyrene, magnetic beads or the like. The DNA, RecA or MutS may be directly labeled by direct bonding or binding of the label to the protein. However, the term “delectably labeled,” whether referring to a protein or DNA, includes “indirect” labeling wherein the “detectable label” is a primary antibody, or any other binding partner, of that protein or DNA, which is directly labeled. Alternatively, the detectable label is a combination of an unlabeled primary antibody (e.g., anti-MutS, anti-RecA, anti-SSB) with a directly labeled secondary antibody specific for the primary antibody.

In the present method, MutS (or its homologue), may be in solution or immobilized to any solid support.

When the RecA or the probe above is labeled, a preferred label is a fluorophore, a chromophore, a radionuclide, biotin, digoxigenin, and wherein association of the probe label with the MutS label or the RecA label with the MutS label, is indicative of the presence of the mutation or the SNP in the test DNA.

When the MutS or MutS homologue is labeled, the label is preferably a fluorophore, a chromophore, a radionuclide, biotin, digoxigenin, or a labeled bead.

A preferred label for RecA or the RecA homologue is a fluorophore.

When the detectable label is a fluorophore a preferred detection method is flow cytometry.

In the above method, the MutS or MutS homologue may be immobilized to a solid support.

In a preferred embodiment the RecA protein is labeled and the detection is of the MutS label associated with the RecA label present in the DNA D loop structures.

In another embodiment, the detectable label of RecA or its homologue is in the form of a labeled primary anti-RecA antibody, or a combination of an unlabeled primary anti-RecA antibody and a labeled secondary antibody specific for the primary antibody.

MutS binding to the duplex portion of the triple strand or D-loop structure stabilizes the structure, allowing use of relatively short oligonucleotides. This allows separate detection of mutations and SNPs which may be close together. Thus, the use of MutS in the present method is an important general improvement over the prior art, e.g., the Sena et al. patents, as it serves as the basis for discriminating between a D loop structure that includes a mutation or SNP and one that does not. This is because D-loop structures that do not bind MutS are those in which the probe and test DNA are perfectly paired, without any mismatches or unpaired bases, a state that favors dissociation of the probe from the test DNA. This has the additional advantage of minimizing background signals.

In the above method, the DNA D loop structure may be further stabilized by the addition, before step (d) above of the single strand DNA binding (SSB) protein (Chase et al., Nucl Acids Res 8:3215-3227 (1980)), or an SSB homologue, which is optionally detectably labeled. When the SSB protein is labeled, the label may be a fluorophore, a chromophore, a radionuclide, biotin, digoxigenin, or a labeled bead, and the association of the SSB label with the MutS label is indicative of the presence of the mutation or the SNP in the test DNA.

Stability of the three strand structure can also be enhanced by allowing DNA synthesis to extend the oligonucleotide. Such extension requires addition of a DNA polymerase and all four deoxynucleotide triphosphates.

In the above method, flow cytometric detection may detect the coincidence of two, three or four labels which are bound to: (a) the MutS and the probe; (b) the MutS and the RecA; (c) the MutS, the RecA and the probe; (d) the MutS and the SSB; (e) the MutS, the SSB, and the probe; or (f) the MutS, the SSB, the probe and the RecA.

The present invention also provides a kit useful for detecting a one or more mutations or polymorphisms in a DNA sample, the kit being adapted to receive therein one or more containers, the kit comprising:

(a) a first container containing a RecA protein, which is optionally detectably labeled;

(b) a second container containing MutS protein which is optionally detectably labeled; and

(c) a third container or plurality of containers containing buffers and reagent or reagents capable of detecting the binding of MutS or the MutS homologue.

Also included is a kit useful for detecting a specific mutation or polymorphism or a specific group of mutations or polymorphisms in a DNA sample, or for examining a specific region or regions of DNA for any mutations or polymorphisms, the kit being adapted to receive therein one or more containers, the kit comprising:

(a) a first container containing RecA protein, or a homologue thereof, which is optionally detectably labeled;

(b) a second container containing MutS protein which is optionally detectably labeled;

(c) a third container or plurality of containers containing a specific oligonucleotide probe or probes, which probes are selected to be complementary to specific sequences in specific regions in the DNA of the sample and which form mismatch-containing or unpaired base-containing heteroduplexes with a mutated or polymorphic sequence or sequences in the specific DNA regions, which probe or probes is or are optionally detectably labeled; and

(d) A fourth container or plurality of containers containing buffers and reagents capable of detecting the binding of MutS or its homologue to specific heteroduplexes formed between the probes and the sample DNA.

### BRIEF DESCRIPTION OF THE DRAWINGS

FIGS. 1-7 are schematic representations of the RecA+MutS mutation/SNP detection method including various detection modalities.

FIG. 1 shows an oligonucleotide “probe” to which is added in Step (1) the RecA (∘) protein. RecA coats the probe to form a “RecA filament.” In Step (2) RecA filament is added to test DNA and allowed to form a triple stranded or “D-loop” structure. In Step (3), the MutS protein is added. If the probe is identical to the test DNA sequence, a perfectly paired duplex (“no mismatch”) is formed and the MutS does not bind (left). If there are one or more sequence differences between the probe and test DNA sequences, a heteroduplex is formed containing one or more mismatches or unpaired bases (“Mismatch (SNP)”) and MutS binds to that heteroduplex.

FIG. 2 shows the final complex formed after Step (3). Here both the probe and MutS are labeled. ×=probe label. ★=MutS label.

FIG. 3 shows the final complex formed after Step (3) when MutS is labeled (★) and RecA is labeled (*)

FIG. 4 shows the final complex formed after Step (3) when the SSB protein (⋄) has been added. The labeled (▾) SSB binds to ssDNA. Here, MutS is also labeled ★

FIG. 5 shows the final complex formed after Step (3) where MutS is labeled and the probe has been labeled by polymerase extension using labeled deoxynucleotide triphosphates -≯≮-≯≮-.

FIG. 6 shows the final complex formed after Step (3) when MutS is immobilized to a solid surface (diagonal lines). In this embodiment, the probe is labeled (×).

FIG. 7 shows the final complex formed after Step (3) when the probe (whose sequence has a mismatch or mispairing with the test DNA) was in the form of two complementary oligonucleotides each of which was “coated” with RecA (∘). MutS is labeled ★.

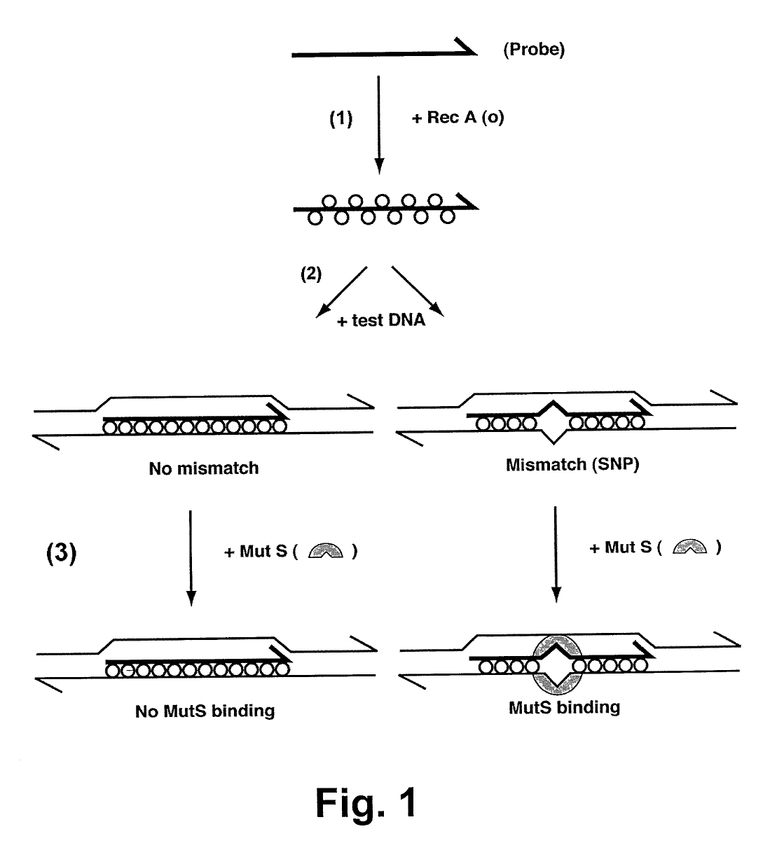

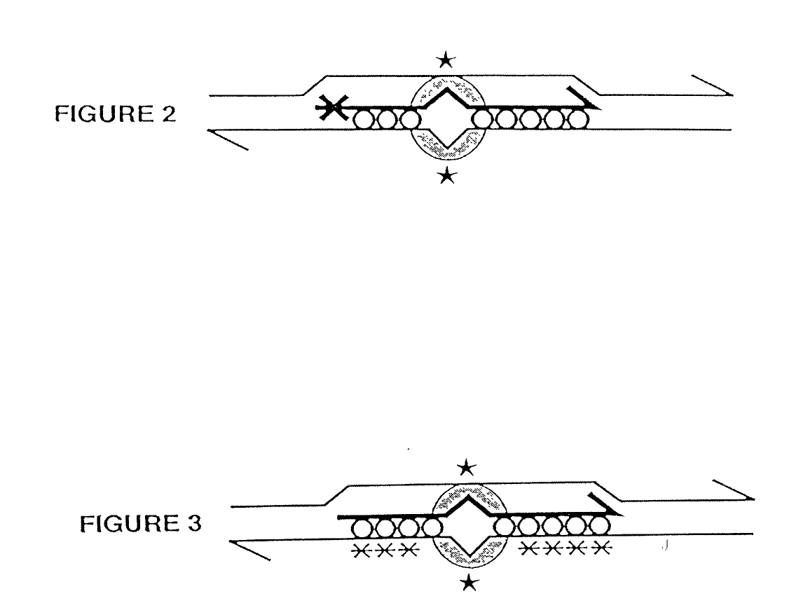

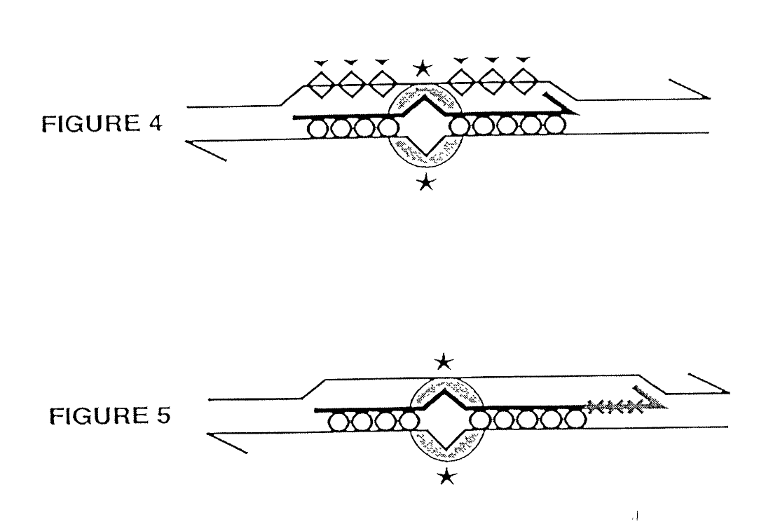

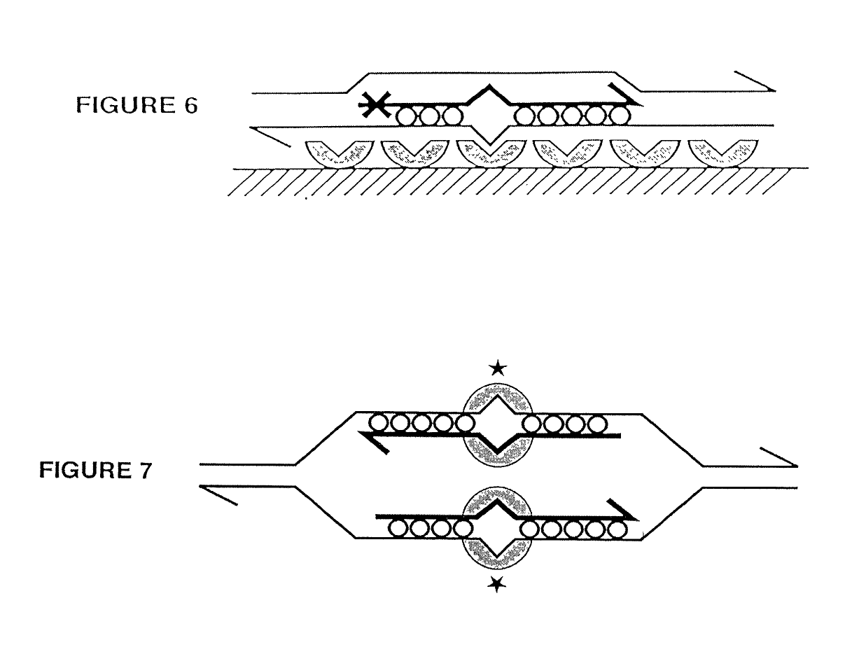

The structure formed is a double D-loop.

### DESCRIPTION OF THE PREFERRED EMBODIMENTS

The present inventors have devised a novel technology for detecting mutations or SNP's using a combination of at least two DNA binding proteins, RecA and MutS. In general, the method employs:

(1) a test DNA molecule, which may be any synthetic, viral, plasmid, prokaryotic or eukaryotic DNA from any source and may be amplified by PCR or any other means;

(2) a DNA probe, which may be any synthetic oligonucleotide, PCR amplicon, plasmid DNA, viral DNA, bacterial DNA or any other DNA of known sequence or of sequence complementary to the test DNA or to a portion thereof,

(3) E. coli RecA or a homologue thereof, as defined below, and

(4) E. coli MutS or a homologue thereof, as defined below, or another mismatch-binding protein from any prokaryotic or eukaryotic species.

As used herein and in the present claims (for the sake of brevity and clarity), the term “MutS,” “RecA” or “SSB” is intended to include either the native or mutant E. coli MutS, RecA or SSB protein, or a “homologue” thereof as defined below. A “homologue” of MutS, RecA, SSB, etc., is a protein that has functional and, preferably, also structural/sequence similarity to its “reference” protein. One type of homologue is encoded by a homologous gene from another species of the same genus or even from other genera. As described below, the above proteins, originally discovered in bacteria, have eukaryotic homologues in groups ranging from yeast to mammals. A functional homologue must possess the biochemical and biological activity of its reference protein, particularly the DNA binding selectivity or specificity so that it has the utility described herein. In view of this functional characterization, use of homologues of E. coli RecA, MutS or SSB proteins, including proteins not yet discovered, fall within the scope of the invention if these proteins have the described DNA binding activity or “improved” binding activity. Nonlimiting examples of improvements include recognition of the C:C mismatch by a MutS homologue, a RecA homologue that binds to shorter DNA molecules, or higher affinity binding of single stranded DNA by a SSB homologue.

“Homologue” is also intended to include those proteins altered by mutagenesis or recombination which has been performed to improve the protein's desired function for use in this invention. These approaches are generally described and well-referenced below. Clearly, it is within the skill of the art to develop such genetically engineered homologues without resorting to undue experimentation. Thus, for example, one would apply these approaches, starting with, for example, DNA encoding a “native” MutS protein (mutagenesis) or two or more DNA molecules each encoding different “native” MutS proteins (recombination), express the gene product, and, using known screening techniques (including the methods of this invention), measure the appropriate DNA binding activity. Hence, even in the absence of specific examples of genetically engineered, improved MutS, RecA or SSB homologues, those skilled in the art are enabled to produce and identify such homologues using only routine experimentation.

Mutagenesis of a protein gene, conventional in the art, is generally accomplished in vivo by cloning the gene into bacterial vectors and duplicating it in cells under mutagenic conditions, e.g., in the presence of mutagenic nucleotide analogs and/or under conditions in which mismatch repair is deficient. Mutagenesis in vitro, also well-known in the art, generally employs error-prone PCR wherein the desired gene is amplified under conditions (nucleotide analogues, biased triphosphate pools, etc.) that favor misincorporation by the PCR polymerase. PCR products are then cloned into expression vectors and the resulting proteins examined for function in bacterial cells.

Recombination generally involves mixing homologous genes from different species, allowing them to recombine, frequently under mutagenic conditions, and selecting or screening for improved function of the proteins from the recombined genes. This recombination may be accomplished in vivo, most commonly in bacterial cells under mismatch repair-deficient conditions which allow recombination between diverged sequences and also increase the generation of mutations. One of the present inventors has developed such methods of protein “evolution” (Radman et al. U.S. Pat. Nos. 5,912,119 and 5,965,415). In addition, Stemmer and colleagues have devised methods for both in vivo and in vitro recombination of diverged sequences to create “improved” proteins. Most involve PCR “shuffling” wherein two diverged sequences are digested and mixed together such that the fragments serve as both primer and template for PCR and, in so doing, combine different segments of the diverged genes, which is, in effect, genetic “recombination.” Frequently, error prone PCR conditions are included to further stimulate generation of novel sequences. Resulting PCR products are cloned into expression vectors, and the resulting proteins are screened for improved function. See, for example, U.S. Pat. Nos. 5,512,463; 5,605,793; 5,81,238; 5,830,721; 5,837,458; 6,096,548; 6,117,679; 6,132,970; 6,165,793; 6,180,406; 6,251,674; 6,277,638; 6,287,861; 6,287,862; 6,291,242; 6,297,053; 6,303,344; 6,309,883; 6,319,713; 6,319,714; 6,323,030; 6,326,204; 6,335,160; 6,344,356.

As noted, homologues of the present invention generally share sequence similarity with their reference protein. To determine the % identity of two amino acid sequences (or of two nucleic acid sequences), the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred method of alignment, Cys residues are aligned. The length of a sequence being compared is at least 30%, preferably at least 40%, more preferably at least 50%, even more preferably at least 60%, and even more preferably at least 70%, 80%, or 90% of the length of the reference sequence (e.g., E. coli MutS or E. coli RecA). The amino acid residues (or nucleotides) at corresponding amino acid (or nucleotide) positions are then compared. When a position in the first sequence is occupied by the same amino acid residue (or nucleotide) as the corresponding position in the second sequence, then the molecules are identical at that position. As used herein amino acid or nucleic acid “identity” is also to be considered amino acid or nucleic acid “homology”. The % identity between the two sequences is a function of the number of identical positions shared by the sequences, taking into account the number of gaps and the length of each gap which need to be introduced for optimal alignment. The comparison of sequences and determination of % identity between two sequences can be accomplished using mathematical algorithms, e.g., the Needleman and Wunsch (J. Mol. Biol. 48:444-453 (1970) algorithm which has been incorporated into the GAP program (see below) using either a Blossom 62 matrix or a PAM250 matrix. A preferred program, “GAP” in the GCG software package, available at http://www.gcg.com, uses a NWSgapdna.CMP matrix and a gap weight of 40, 50, 60, 70, or 80 and a length weight of 1, 2, 3, 4, 5, or 6. In another approach, the % identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

The nucleic acid or protein sequence of a particular MutS, RecA or SSB protein can further be used as a “query sequence” to perform a search against public databases, for example, to identify other family members or related sequences. Such searches can be performed using the NBLAST and XBLAST programs (version 2.0) of Altschul et al. (1990) J. Mol. Biol. 215:403-410. To obtain gapped alignments for comparison purposes, Gapped BLAST can be utilized as described in Altschul et al. (1997) Nucl. Acids Res. 25:3389-3402. When using BLAST and Gapped BLAST, the default parameters of the respective programs can be used. See http://www.ncbi.nlm.nih.gov. For example, BLAST nucleotide searches can be performed with the NBLAST program, score=100, wordlength=12 to obtain nucleotide sequences homologous to a query MutS, RecA or SSB coding nucleic acid sequence. BLAST protein searches can be performed with the XBLAST program, preferably set at score=50, wordlength=3, to obtain amino acid sequences homologous to a query MutS, RecA or DNA protein molecule (e.g., wild-type sequence from E. coli).

Thus, a preferred homologue of an E. coli MutS protein, an E. coli RecA protein or an E. coli SSB protein has, first and foremost, the functional activity of native E. coli MutS (or RecA or SSB), or even improved activity over the native protein as noted above. A preferred homologue also shares sequence similarity with the native E. coli protein, when determined as above, of at least about 20% (at the amino acid level), preferably at least about 40%, more preferably at least about 60%, even more preferably at least about 70%, even more preferably at least about 80%, and even more preferably at least about 90%.

At least 65 RecA genes from different bacteria have been cloned and sequenced (Sandler, S J, et al., Nucl Acids Res 24:2125-2132 (1996); Roca, A I, et al., Crit Rev Biochem Mol Biol 25:415-456 (1990); Eisen, J A, J. Mol. Evol. 41:1105-1123 (1995); Lloyd, A T, et al., J. Mol. Evol. 37:399-407 (1993)). RecA homologues, known as RadA, have been identified in three archaean species (Sandier et al., supra; Seitz, E M, et al., Genes Dev. 12:1248-1253 (1998)). Eukaryotic homologues of RecA have been identified in every eukaryotic species examined; the prototype eukaryotic RecA homologue is the yeast Rad51 protein (Seitz et al., supra; Bianco, P R, et al., Frontiers Biosci. 3:570-603 (1998)). Therefore, any homologue of E. coli RecA which, like the E. coli protein, forms DNA filaments for initiation of genetic recombination as well as any functional form that has been mutated or evolved in vivo or in vitro is included within the scope of the present invention.

Examples of E. coli MutS homologues are the MutS protein of Salmonella typhimurium (Lu et al., supra; Haber, L T, et al., J. Bacteriol. 170:197-202 (1988; Pang, P P, et al., J. Bacteriol. 163:1007-1015 (1985); and the HexA protein of Streptococcus pneumoniae (Priebe S D, et al., J. Bacteriol. 170:190-196 (1988); Haber et al., supra). In addition, eukaryotic homologues of MutS or HexA can also be used, such as those encoded by the homologous sequences identified in yeast, human, mouse, frog or hamster DNA (Shimada, T, et al., J. Biol. Chem. 264:20171 (1989); Linton, J, et al., Mol. Cell. Biol. 7:3058-3072 (1989); Fujii, H. et al., J. Biol. Chem. 264:10057 (1989)). The homology between MutS homologues in prokaryotic and eukaryotic species is illustrated in Reenan, R A et al, Genetics 132:963-973 (1992), where the E. coli MutS nucleotide sequence is shown to be highly homologous in one region to S. typhimurium MutS, S. pneumoniae hexA, mouse Rep-1, and human DUC-1. PCR primers which successfully led to the cloning of Saccharomyces cerevisiae (yeast) homologues of MutS, named MSH1 and MSH2, were based on this homology. Reenan et al., supra, showed the amino acid sequence homology between yeast MSH1, MSH2 and E. coli, S. typhimurium and S. pneumoniae MutS homologues. New, L. et al., Mol. Gen. Genet. 239:97-108 (1993) disclosed another yeast gene, MSH3, which is a homologue of eukaryotic MutS and indicates the most conserved sequences among MutS, HexA and mouse REP-3. A search for a new yeast gene based on this sequence homology led to discovery of yeast MSH3. Fishel, R. et al., Cell 75:1027-1029 (1993) described the cloning of a another human MutS homologue (hMSH2) using for PCR the homologous sequences from other MutS homologues as described by Reenan et al, supra.

Therefore, any homologue of E. coli MutS which, like the E. coli protein, recognizes DNA mismatches (single base mismatches or several unpaired bases) as well as any functional form version that has been mutated or evolved in vivo or in vitro is included within the scope of the present invention.

Both MutS and RecA function in vitro. MutS is the basis of the Gene Check Immobilized Mismatch Binding Protein (IMBP) mutation detection technology which is currently being used commercially to genotype sheep for scrapie susceptibility (Wagner et al., supra; Debbie et al., supra; U.S. Pat. Nos. 6,027,877, 6,114,115, 6,120,992, 6,329,147, all of which references are incorporated by reference in their entirety).

RecA forms a three stranded structure in vitro along sequence stretches as short as 15 nucleotides (Ferrin et al., 1991, supra). Combining the activities of RecA and MutS, creates a most powerful mutation/single nucleotide polymorphism (SNP) detection system in which RecA-coated ssDNA catalyzes formation of a three strand (or “D-loop”) structure without the need for prior denaturation of the test dsDNA. The D-loop will contain mismatches or unpaired bases when the sequences of the probe DNA and the test DNA are not identical. These mispaired or unpaired bases can be recognized by MutS (or any MutS homologue from any species or any other mismatch binding protein) MutS binding will stabilize the D-loop structure. Thus, the combination of MutS with RecA mediated D-loop formation allows formation of very small, stable D-loops (when such D-loops contain mispaired or unpaired bases), which in turn allows separate examination of short DNA intervals for mutations and SNPs as well as small scale scanning for known (and unknown) mutations and SNPs. Such examination is neither suggested nor supported by U.S. Pat. Nos. 5,273,881 and 5,670,316, cited above.

The combined “RecA/MutS” method can be used with a variety of platforms some which allow mutation/SNP detection without the need for PCR amplification of the test DNA.

RecA/MutS System with Flow Cytometric Detection of MutS-RecA Co-Localization

In one preferred embodiment, the present system employs:

(1) labeled RecA and MutS;

(2) specific probe oligonucleotides that are detectably labeled for detection by flow cytometry; and

(3) flow cytometric detection of the labels.

Probe specificity derives from the probe's sequence. A probe is designed to be complementary to the “normal” or wild type, non-polymorphic sequence of, the site or region of interest as well as the flanking region. When a mutation or polymorphism is present the probe/test heteroduplex will contain one or a few mispaired or unpaired bases. In the absence of a mutation or polymorphism, probe/test heteroduplex will be perfectly paired.

Formation or stabilization of the D-loop formed by the RecA filaments and test DNA may be further enhanced by the addition of single strand binding (SSB) protein from E. coli or a homologue of this protein from another species or by allowing DNA polymerase catalyzed extension of the probe DNA using the test DNA as template.

In this method detection of mutations and SNPs is accomplished by detecting the co-localization of either (a) RecA and MutS, (b) probe DNA and MutS or (c) RecA, MutS and probe DNA. Alternatively, labeled SSB, or a homologue can be used to label D-loop structures by binding to the single stranded portion. In this instance, co-localization of SSB signal with MutS signal is indicative of the presence of one or more mismatches or unpaired bases in the duplex portion of the D-loop.

The DNA probe may be of any length but is preferably an oligonucleotide, more preferably a synthetic oligonucleotide, of about 30-60 bases in length.

The probe is specific for a particular mutation or polymorphism, or specific for a particular genetic region that is being examined for the presence of a known or unknown mutation or SNP.

The test DNA may be of any length (up to an entire chromosome) and can be either genomic or plasmid DNA or a PCR amplicon.

Labeling of MutS, RecA and SSB proteins can be accomplished via a variety of well established methods: The proteins can be directly labeled with fluorophores or fluorescent labels, including, but not limited to, fluorescein (and derivatives), 6-Fam, Hex, tetramethylrhodamine, cyanine-5, CY-3, allophycocyanin, Lucifer yellow CF, Texas Red, rhodamine, Tamra, Rox and Dabcyl. Indirect labeling utilizes, for example, “primary” antibodies (monoclonal or polyclonal) specific for MutS or RecA which can be labeled (e.g., with fluorophores). Alternatively, secondary antibodies, e.g., anti-immunoglobulin such as anti-isotype antibodies, specific for the primary anti-MutS and anti-RecA antibodies can be labeled (e.g., with fluorophores).

In another embodiment, the proteins (MutS or RecA, primary antibodies or secondary antibodies) are biotinylated. The biotin is then bound by fluorescent avidin (or streptavidin). Alternatively, streptavidin (which is multivalent) may first be bound to the biotin of the biotinylated protein, and then bound to other fluorescently-labeled biotinylated compounds. The proteins can be attached to fluorescent microbeads, or microbeads to which is attached a different detectable label. Attachment of proteins to fluorescent microbeads may be by any methods well known in the art, including, but not limited to, direct adsorption to polystyrene or other beads, covalent linkage via carboxyl, amino, tosyl or other groups, binding via biotin/avidin or streptavidin interaction (requires biotinylation of the protein) and binding to immobilized antibody. The labeling of MutS by attaching it to a labeled bead is functionally equivalent to immobilization (Wagner et al., supra; Debbie et al., supra) and will, therefore, enhance MutS (or other mismatch binding protein) function similarly to the effect observed when MutS is immobilized to nitrocellulose or polystyrene.

It is also possible to label MutS and the DNA probe (instead of, or in addition to, labeling of RecA). The probe may be labeled directly with a fluorophore or with a compound such as biotin or digoxigenin and detecting the adducts by conventional methods. The probe is detected by measurement of fluorescence, color luminescence or any other method suitable for the label that has been selected.

Signal amplification can be introduced, for example, by using (a) labeled secondary antibodies or (b) avidin- or streptavidin-coated microbeads to bind biotin labeled probe. This will result, for example, in multiple biotinylated probes being bound, which will, in turn, be bound by MutS molecules (if the probe/test DNA complex contains one or more mismatches or unpaired bases) and thereby, greatly increasing the signal.

The procedure is carried out by mixing the probe with RecA, MutS and the test DNA under conditions appropriate for formation of RecA filaments, D-loops or triple strand structures. RecA filament formation can be accomplished, for example, in a Tris-HCl or Tris-acetate buffer, (20-40 mM, pH 7.4-7.9) with MgCl₂ or Mg acetate (1-4 mM), dithiothreitol (0.2-0.5 mM), and ATP or ATP[γ-S] (0.3-1.5 mM). If ATP is used, an ATP regenerating system consisting of phosphocreatine and creatine kinase may be included. RecA and probe are generally added at a molar ratio of 0.1-3 (RecA to nucleotides). If the probe is double stranded, it must first be denatured before RecA coating. Incubation is at room temperature or, preferably, 37° C., for 5-30 min. D-loop or triple strand structure formation involves adding RecA filaments to double stranded DNA and incubating, preferably at 37° C., for about 15 min-2 hrs. It is also possible to form RecA filaments and do homology searching in a single reaction vessel, i.e., to mix RecA with oligonucleotides and double stranded DNA at the same time. See, for example, Rigas et al., supra; Honigberg, S M, et al., Proc Natl Acad Sci USA 83:9586-9590 (1986); any of the Ferrin et al. publications (supra).

Mismatch binding by MutS is accomplished by adding the MutS to the double stranded test DNA at or before the time RecA or RecA filaments are added. MutS may be in solution or immobilized. Generally, 0.1 ng-0.5 μg of MutS is added. See, for example, Lishanski et al., supra; Wagner et al., supra; Debbie et al., supra; U.S. Pat. Nos. 6,027,877, 6,114,115, 6,120,992, 6,329,147; Gifford, U.S. Pat. No. 5,750,335; Jiricny et al., supra.

Following incubation to allow homology search by the RecA filament and binding of mismatched or unpaired bases by MutS, the mixture is preferably analyzed by flow cytometry in the case of fluorescent labeling. The flow cytometer is set to detect as a signal the simultaneous presence of both labels (that on the MutS and that on the RecA and/or probe) or the presence of a “third” color created by the juxtaposition of the two (or three) labels. The presence of such signals is an indication of the presence in the sample of sequences differing from the probe by one or a few single mismatches or unpaired bases.

The power of the RecA/MutS method described herein is that the background signals are very low, and RecA+MutS (or MutS+DNA probe or MutS+RecA+DNA probe) will be found together only under conditions in which RecA-coated oligonucleotide probe has bound to test DNA in a way that creates a heteroduplex with a mismatched or unpaired base.

Although MutS would be expected to bind to other mismatches found in the test DNA, these should be rare. This is so because in this method the test DNA is never denatured and annealed, a process which can cripple current MutS-based mutation detection technologies because of the random mismatches created by homologous (or nearly homologous) DNA annealing and by incomplete annealing which results in regions of ssDNA that can form mismatch-containing secondary structure. RecA has the capacity to coat any ssDNA in the sample, but, for the reasons described above, stretches of ssDNA will be very rare.

The sequence of interest will, in general, be present only once per chromosome. It is therefore a simple matter to set the flow conditions to detect each occurrence of RecA-MutS or probe DNA-MutS juxtaposition. To overcome random MutS binding (if it occurs), DNA fragment size can be reduced, by shearing or nuclease digestion. The effect of this reduction is to minimize the likelihood of random MutS binding in the same fragment in which the probe and RecA bind.

In another generally applicable embodiment of this invention, two complementary DNA probes can be employed instead of one. These probes are preferably precoated separately with RecA before being added to the test DNA in order to minimize their self-annealing. The two complementary, ssDNA probes will bind to both strands of test DNA in the region of interest, thereby: (1) helping stabilize the D-loop structure by forming a probe-length double duplex, (2) assuring detection of all mutant/wild type pairings, including those arising from G to C or C to G transversions, and (3) increasing signal, particularly from poorly recognized mismatches. The assurance of recognition in (2), above, is particularly important because C:C mismatches are not detected in vivo or in any mismatch detection methods developed to date, whereas the reciprocal G:G mismatches are always well detected. The present inventors and their colleagues have discovered that increasing the number of mismatches in a single heteroduplex fragment increases binding by MutS and each duplex region in a double duplex structure will contain a mismatch.

In another generally applicable embodiment of this invention, MutS may be immobilized, and either the probe of the RecA may be detectably labeled. In this embodiment, binding of the probe or RecA to immobilized MutS is indicative of one or more mismatches or unpaired bases in the D-loop structure formed between the probe and test DNA.

In another generally applicable embodiment, test dsDNA is immobilized to a solid support, to allow detection of mutations and SNPs by labeling, as described above. Here, the preferred test DNA is PCR-amplified DNA. Immobilization of amplified DNA can be accomplished by using a 5′ label, such as biotin, or a carboxy or amino group on one of the primers. Amplified DNA with a 5′ biotin label can be immobilized to a solid support via avidin or streptavidin binding. Probe DNA is coated with RecA and mixed with the immobilized DNA or mixed with the DNA prior to immobilization under conditions that allow triple strand or D-loop structure formation. Labeled MutS, or a homologue, is added either before, after or during triple strand or D-loop structure formation. Binding of MutS (or its homologue) to the immobilized DNA indicates the presence of one or more mismatches or unpaired bases in the triple strand or D-loop structure. This embodiment is ideally suited for use in microarray (DNA chip) applications.

In another generally applicable embodiment, oligonucleotide probes are prepared with a 5′ adduct to allow immobilization of the probe/test complex as in Rigas et al., supra. The adduct may be a biotin moiety, a specific oligonucleotide or any other adduct that would allow specific retrieval of the oligonucleotide. The probe is mixed with RecA to form RecA filament and then mixed with test DNA to form specific D-loop structures. The probes may contain an additional detectable label or may be labeled after D-loop formation by allowing the annealed oligonucleotides to be extended by DNA polymerase using labeled nucleotide triphosphates or nucleotide triphosphate analogues. In this case, only those oligonucleotides which form D-loop structures will be labeled, which will reduce, even further, any background signal. Further specificity can be obtained by using nucleotide terminators, such as dideoxy- or acyclo-nucleotide triphosphates in a mix of all four terminators wherein label is associated only with the terminator which is complementary to the first base in the test DNA beyond the 3′ end of the oligonucleotide.

The 5′ label of the oligonucleotide can be used to immobilize the D-loop structure to any solid support. Association of the MutS label with the 3′ label of the oligonucleotide indicates the presence of one or more mismatches or unpaired bases in the D-loop region and will be diagnostic of the presence or absence of a specific sequence in the test DNA. If the solid support is a microtiter plate, the ratio of MutS signal to oligonucleotide signal will be characteristic of the genotype of the test DNA. For example, a very high ratio (extensive MutS binding) indicates homozygosity for the test genotype with sequence different than the probe, wherein heteroduplexes in the D-loop contain mismatches or unpaired bases. A low ratio (little of no MutS binding) indicates homozygosity for the test genotype with sequence identical to the probe, wherein heteroduplexes in the D-loop are perfectly paired. An intermediate ratio (approximately half of the high ratio), indicates a heterozygous genotype, wherein approximately half the heteroduplexes in D-loops are perfectly paired and half contain mismatches or unpaired bases.

In applying the present method to clinical diagnosis, it is possible to perform multiple assays with a small blood sample (because 1 μl of blood contains about 10⁴ copies of each sequence). For the sequence being detected, the number of positive signals in a sample is an indication of the genotype, as low or no signal indicates that the sample is homozygous for a sequence perfectly complementary to the probe. A very high signal indicates homozygosity for a sequence differing from the probe by one or a few single nucleotide substitutions or one or a few unpaired bases (as determined by the recognition properties of MutS or its homologue). An intermediate signal indicates heterozygosity for the sequence of interest.

For routine mutation detection, standards (i.e., known genotypes) are run with each batch of test samples to provide a standard curve for genotype determination. Of course, standard curve formation and genotype determination depend on accurate test DNA quantitation. Thus, when RecA-catalyzed D-loop formation, MutS binding and flow cytometric signal detection are all efficient, so that, e.g., 5,000-20,000 sequences are sufficient for a genotype determination, as many as 1000 or more separate assays can be performed on a single ml of blood.

This technology is ideally suited to multiplexing wherein several sites in a single sample of genomic, plasmid or amplified DNA are interrogated simultaneously. In this application, specific probes are designed with adducts, most preferably 5′ oligonucleotide adducts that allow individual probes+test DNA (D-loop or triple strand structures) to be separately isolated from a mixture of many probes and test DNA. When 5′ oligonucleotides are used, isolation involves annealing the specific oligonucleotide adducts to immobilized oligonucleotides of complementary sequence. Probe label for detection can be added to the 3′ opposite end of the probe from the isolation moiety or can be added by polymerase during the reaction (see below).

It is also possible to run multiple sequential tests on a single sample by deproteinizing the sample after each flow cytometry run. Removal of RecA and MutS will cause any three stranded structures to fall apart. The oligonucleotide probe can then be removed by passing the sample through a mini DNA binding column (which does not retain short oligonucleotides). The test DNA can then be recycled through the entire process with a new probe. This entire process can easily be automated using methods and technologies well-known in the art.

Example of RecA/MutS System Combined with Immobilized MutS

Because RecA facilitates heteroduplex formation without requiring denaturation of test DNA, the present RecA/MutS system can be used as an enhancement for virtually any mutation/SNP detection method which depends on formation of heteroduplex DNA. This includes: (1) gel shift assays, (2) filter binding assays, (3) mismatch cleavage assays and (4) Immobilized Mismatch Binding Protein (IMBP) assays. IMBP assays, developed by one of the present inventors, utilizes components of the present RecA/MutS system, including labeled probe DNA and MutS (immobilized).

However, the most successful IMBP assay formats require probes of length equal to the test DNA (generally a PCR amplicon). Combining the RecA/MutS system with the IMBP assay accomplishes the following: (1) eliminates the need to denature test DNA, (2) allows the use of longer PCR products than were usable with previous IMBP assay formats, (3) allows the replacement of long probes (which are generally produced by PCR amplification of cloned sequences) with shorter synthetic oligonucleotide probes, and (4) allows a single PCR product to be examined at several different sites along its sequence by using a combination of short oligonucleotide probes.

One major problem afflicting mutation detection assays that require PCR amplification of test DNA is the introduction of errors during amplification. PCR is well known to be a low fidelity process when compared to in vivo DNA replication Errors introduced in each amplification cycle are propagated throughout the entire process and, because the errors are, in general, introduced at random positions, they will all form mismatches when the PCR amplicon is denatured and annealed. Because the number of “errors per fragment” is of primary concern and because the likelihood of an error in a given fragment clearly depends on fragment length, the frequency of PCR errors limits the length of PCR fragments that can be used in mutation detection assays.

The RecA/MutS system of the present invention minimizes or eliminates these concerns about PCR errors by completely eliminating the need to amplify or denature the test DNA. Therefore, even when amplification is employed, the only errors that will affect the assay are those created in the final PCR cycle (because earlier errors will be copied correctly in the final PCR cycle and the molecules will not, thereafter, be denatured. Moreover, probe annealing to the test DNA will not introduce a significant number of mismatches or unpaired bases since the probes of the present invention are preferably very short (i.e., about 20-60 nucleotides).

Conditions described above are employed when applying the RecA/MutS method to IMBP assays. The probe can be of any length and from any source, but is preferably a synthetic oligonucleotide or a pair of complementary oligonucleotides (see above) of about 20-60 nucleotides in length. Again, the probe is specific for (1) a particular mutation or polymorphism or (2) specific for a particular region being examined for the presence of known or unknown single nucleotide polymorphism.

When a pair of probes is used (see above), they are preferably mixed individually with RecA. Formation of the RecA filaments is permitted to take place either in the presence of test DNA or before mixing with test DNA.

Test DNA may be from any source, preferably genomic DNA or PCR amplified DNA.

D-loops (or double duplexes when a pair of complementary probes is used) may be formed before mixing with (or in the presence of) immobilized MutS (or other MBP). Immobilization may be to any solid support or carrier. By “solid support” or “carrier” is intended any support capable of binding a protein while permitting washing without dissociating from the protein. Well-known supports or carriers include, but are not limited to, natural cellulose, modified cellulose such as nitrocellulose, polystyrene, polypropylene, polyethylene, polyvinylidene difluoride, dextran, nylon, polyacrylamide, and agarose or Sepharose®. Also useful are magnetic beads. The support material may have virtually any possible structural configuration so long as the immobilized MBP is capable of binding to the target nucleic acid molecule. Thus, the support configuration can include microparticles, beads, porous and impermeable strips and membranes, the interior surface of a reaction vessel such as test tubes and microtiter plates, and the like. Those skilled in the art will know many other suitable carriers for binding the MBP or will be able to ascertain these by routine experimentation.

As above, SSB protein is optionally used to facilitate D-loop formation and increase D-loop stability. As noted above, MutS stabilizes D-loop structures that include a mismatch or unpaired bases.

Detection of MutS-bound DNA is normally accomplished by using labeled probes. Probes may be labeled with any fluorophore, chromophore, radionuclide or luminescer prepared by any labeling method, including those described above. Probe labeling may also be accomplished by polymerase mediated extension of the oligonucleotide in the D-loop structure using labeled nucleotides or nucleotide analogues. Labeling of RecA, particularly via a labeled anti-RecA antibody, would amplify the signal so that genomic DNA can be tested without prior amplification.

It is difficult to overstate the power of the RecA/MutS method. It is rapid, works with small samples and can readily be adapted to clinical applications for diagnostic genotyping and mutation/SNP detection. Perhaps the most important distinguishing advantage of the present invention is its complete independence from DNA amplification (i.e., PCR).

Kits

The present invention is also directed to kit or reagent systems useful for practicing the methods described herein. Such kits will contain a reagent combination comprising the essential elements required to conduct an assay according to the methods disclosed herein. The reagent system is presented in a commercially packaged form, as a composition or admixture where the compatibility of the reagents will allow, in a test device configuration, or more typically as a test kit, i.e., a packaged combination of one or more containers, devices, or the like holding the necessary reagents, and usually including written instructions for the performance of assays. The kit of the present invention may include any configurations and compositions for performing the various assay formats described herein.

Kits containing RecA, MutS and, where applicable, antibodies and/or SSB, are within the scope of this invention. In one embodiment, a kit of this invention designed to allow detection of specific mutations and/or polymorphisms or mutations and/or polymorphisms in specific regions of target DNA, includes oligonucleotides or other probes specific for (a) selected mutations and/or (b) SNPs, or (c) specific region or regions of target DNA (to allow scanning of regions for any mutations or polymorphisms, known or unknown). The probes may be labeled as described above. The kits also include labeled MutS, or antibodies allowing detection of MutS, which may be immobilized to a solid support or carrier or provided in immobilizable form with separate carrier; RecA, which may be labeled; and a plurality of containers of appropriate buffers and reagents.

Another kit, is designed to allow ends users to design their own probes for detection of mutations and/or polymorphisms or to scan a DNA region of their choice; such a kit contains all of the above described reagents except probe DNA.

The references cited above are all incorporated by reference herein, whether specifically incorporated or not.

Having now fully described this invention, it will be appreciated by those skilled in the art that the same can be performed within a wide range of equivalent parameters, concentrations, and conditions without departing from the spirit and scope of the invention and without undue experimentation.

What is claimed is:

1. A method for detecting a mutation and/or a SNP in a double-stranded test DNA molecule, comprising:
(a) providing a single stranded DNA probe which is optionally detectably labeled, which probe has (i) a known nucleotide sequence or (ii) a sequence complementary to the sequence of at least a part of the test DNA;
(b) contacting the probe with a RecA protein which is optionally detectably labeled, to form a RecA filament;
(c) contacting the RecA filament with the test DNA, thereby forming a three stranded DNA D-loop structure in the test DNA, which D-loop structure comprises the probe and the two strands of the test DNA;
(d) contacting the DNA D-loop structure with a MutS protein which is optionally detectably labeled, wherein the MutS binds to one or more base pair mismatches or unpaired bases present in the duplex portion of D-loop structure;
(e) detecting the presence of MutS bound to the DNA D loop structure,
wherein the presence of the bound MutS is indicative of the presence of the mutation or the SNP in the test DNA.

2. A method for detecting a mutation and/or a SNP in a double-stranded test DNA molecule, comprising:
(a) providing a probe comprising two complementary single stranded oligonucleotides which are optionally detectably labeled, which probe has a known nucleotide sequence or a sequence complementary to the sequence of at least a part of the test DNA;
(b) contacting each of the oligonucleotides in single stranded form with a RecA protein, which is optionally detectably labeled, to form RecA filaments,
(c) contacting the RecA filaments with the test DNA, thereby forming a four stranded DNA structure in the test DNA, which structure comprises the probe and the two strands of the test DNA and wherein the probe strands are annealed with test DNA strands;
(d) contacting said DNA structure with a MutS protein which is optionally detectably labeled, wherein the MutS binds to one or more base pair mismatches or unpaired bases present in the four stranded DNA structure;
(e) detecting the presence of the MutS bound to the DNA structure
wherein the presence of the bound MutS is indicative of the presence of the mutation or the SNP in the test DNA.

3. A method for detecting a mutation and/or a SNP in a double-stranded test DNA molecule, comprising:
(a) providing a single stranded DNA probe which is optionally detectably labeled, which probe has (i) a known nucleotide sequence or (ii) a sequence complementary to the sequence of at least a part of the test DNA molecule;
(b) contacting the probe with a RecA protein which is optionally detectably labeled, to form a RecA filament,
(c) contacting the RecA filament with the test DNA, thereby forming a three stranded DNA D-loop structure in the test DNA, which structure comprises the probe and two strands of the test DNA;
(d) contacting the DNA D-loop structure with immobilized MutS which binds to one or more base pair mismatches or unpaired bases present in the duplex portion of the D-loop structure;
(e) detecting the presence immobilized probe DNA or RecA bound to the MutS,
wherein the presence of the bound probe DNA or RecA is indicative of the presence of the mutation or the SNP in the test DNA.

4. A method for detecting a mutation and/or a SNP in a double-stranded test DNA molecule, comprising:
(a) providing a probe comprising two complementary single stranded oligonucleotides which are optionally detectably labeled, which probe has a known nucleotide sequence or a sequence complementary to the sequence of at least a part of the test DNA;
(b) contacting each of the probe oligonucleotides in single stranded form with a RecA protein, which is optionally detectably labeled, to form RecA filaments,
(c) contacting the RecA filaments with the test DNA, thereby forming a four stranded DNA structure in the test DNA, which structure comprises the two strands of the test DNA to each of which is annealed a probe oligonucleotide strand; and
(d) contacting said DNA structure with immobilized MutS which binds to one or more base pair mismatches or unpaired bases present in said four stranded DNA structure,
thereby detecting the mutation and/or SNP.

5. The method of any of claims 1-4, wherein the mutation being detected is a single nucleotide substitution or the addition or deletion of 1-4 nucleotides.

6. The method of any of claims 1-4, wherein the test DNA molecule is selected from the group consisting of prokaryotic genomic DNA, eukaryotic genomic DNA, cDNA, viral DNA, plasmid DNA, and a DNA fragment amplified by PCR or by another amplification method.

7. The method of any of claims 1-4, wherein the probe is selected from the group consisting of:
(a) a synthetic oligonucleotide;
(b) a recombinant oligonucleotide;
(c) an oligonucleotide obtained by denaturing, and, optionally cleaving, a double stranded DNA molecule.

8. The method of claim 7, wherein the oligonucleotide has a length of about 20 to about 60 nucleotides.

9. The method of any of claims 1-4, wherein:
(i) the probe and the MutS are labeled;
(ii) the label is a fluorophore, a chromophore, a radionuclide, biotin or digoxigenin; and
(iii) association of the probe label with the MutS label is indicative of the presence of the mutation or the SNP in the test DNA.

10. The method of any of claims 1-4, wherein the RecA protein is from E. coli.

11. The method of any of claims 1-4, wherein
(i) the RecA and MutS are labeled;
(ii) the label is a fluorophore, a chromophore, a radionuclide, biotin or digoxigenin; and
(iii) association of the RecA label with the MutS label is indicative of the presence of the mutation or the SNP in the test DNA.

12. The method of claim 1 or 2 wherein the MutS is immobilized to a solid support.

13. The method of claim 1 or 2 wherein the detectable MutS label is a fluorophore, a chromophore, a radionuclide, biotin, digoxigenin, a detectably labeled bead, a detectable labeled anti-MutS antibody, or a combination of an unlabeled anti-MutS antibody and a detectably labeled secondary antibody specific for the anti-MutS antibody.

14. The method of claim 1 or 2 wherein the RecA protein is labeled and the detection is of the MutS label associated with the RecA label present in the DNA D loop structures.

15. The method of any of claims 1-4, wherein the detectable RecA label is in the form of a detectably labeled primary anti-RecA antibody, or a combination of an unlabeled anti-RecA antibody and a detectably labeled antibody specific for the anti-RecA antibody.

16. The method of any of claims 1-4, wherein one or more of the detectably labeled probe, the detectably labeled RecA and/or the detectably labeled MutS is labeled with a fluorophore.

17. The method of any of claims 1-4, wherein the detecting is by flow cytometry.

18. The method of claim 16 wherein the detecting is by flow cytometry.

19. The method of claim 1 or 3 wherein the DNA D loop structure is stabilized by the addition, before step (d), of SSB protein which is optionally detectably labeled.

20. The method of claim 19, wherein,
(i) the SSB protein is labeled with a detectable label;
(ii) the label is a fluorophore, a chromophore, a radionuclide, biotin, digoxigenin, a labeled anti-SSB antibody, or a combination of an unlabeled anti-SSB antibody and a labeled secondary antibody specific for the anti-SSB antibody; and
(iii) association of the SSB label with the MutS label is indicative of the presence of the mutation or the SNP in the test DNA.

21. The method of claim 1 or 3 wherein the detecting is by flow cytometry which detects the coincidence of two, three or four labels which are bound to:
(a) MutS and the probe;
(b) MutS and RecA;
(c) MutS, RecA and the probe;
(d) MutS and SSB;
(e) MutS, SSB and the probe; or
(f) MutS, SSB, the probe and RecA.

22. The method of any of claims 1-4 wherein the probe is labeled by polymerase extension using labeled deoxynucleotide triphosphates or nucleotide terminators.

23. The method of claims 1 or 2, wherein the test DNA is immobilized to a solid support.

24. The method of claim 1 or 2, wherein the probe is bonded to an adduct that allows immobilization of the probe following formation of said D-loop structure.

25. The method of claim 24, wherein the adduct is an oligonucleotide.

26. The method of claim 24, wherein the adduct is biotin or digoxigenin.

27. A kit useful for detecting a one or more mutations or polymorphisms in a DNA sample, the kit being adapted to receive therein one or more containers, the kit comprising:
(a) a first container containing a RecA protein which is optionally detectably labeled;
(b) a second container containing MutS protein which is optionally detectably labeled; and
(c) a third container or plurality of containers containing buffers and reagent or reagents capable of detecting bound MutS.

28. A kit useful for detecting a specific mutation or polymorphism or a specific group of mutations or polymorphisms in a DNA sample or for examining a specific region or regions of DNA for any mutations or polymorphisms, the kit being adapted to receive therein one or more containers, the kit comprising:
(a) a first container containing RecA protein which is optionally detectably labeled;
(b) a second container containing MutS protein which is optionally detectably labeled;
(c) a third container or plurality of containers containing a specific oligonucleotide probe or probes, which probes are selected to be complementary to specific sequences in specific regions in the DNA of the sample and which form mismatch-containing or unpaired base-containing heteroduplexes with a mutated or polymorphic sequence or sequences in the specific DNA regions, which probe or probes is or are optionally detectably labeled; and
(d) A fourth container or plurality of containers containing buffers and reagents capable of detecting MutS when it is bound to specific heteroduplexes formed between the probes and the sample DNA.
